(19) United States
(12) Patent Application Publication
(10) Pub. No.: US 2016/0087219 A1
(43) Pub. Date: Mar. 24, 2016

(54) LIGHT STABILIZER, ORGANIC PHOTOVOLTAIC CELL CONTAINING THE SAME AND METHOD FOR PREPARING THE SAME

(71) Applicant: KOREA INSTITUTE OF SCIENCE AND TECHNOLOGY, Seoul (KR)

(72) Inventors: Bong Soo KIM, Seoul (KR); Hong Gon KIM, Seoul (KR); Min Jae KO, Seoul (KR); Doh-Kwon LEE, Seoul (KR); Jin Young KIM, Seoul (KR); Hae Jung SON, Seoul (KR); Na Ra SHIN, Seoul (KR)

(21) Appl. No.: 14/581,007

(22) Filed: Dec. 23, 2014

(30) Foreign Application Priority Data
Sep. 18, 2014 (KR) 10-2014-0124089

(51) Int. Cl. H01L 51/00; C07C 211/55; C07C 211/58

(52) U.S. Cl. CPC H01L 51/0059; C07C 211/58; C07C 211/55; H01L 51/006; H01L 51/0052; H01L 51/44

(57) ABSTRACT

The present disclosure relates to an additive for improving the light stability of a conjugated polymer, a method for preparing the same and an organic photovoltaic cell containing the same. Since the additive of the present disclosure improves the light stability of a conjugated polymer, it can be used for an organic photovoltaic (OPV) cell device and can also be usefully used for an organic optoelectronic device using a conductive polymer, such as an organic photodiode (OPD), an organic thin-film transistor (OTFT), an organic light-emitting diode (OLED), etc.

### CROSS-REFERENCE TO RELATED APPLICATIONS

This application claims priority under 35 U.S.C. §119 to Korean Patent Application No. 10-2014-0124089 filed on Sep. 18, 2014 in the Korean Intellectual Property Office, the disclosure of which is incorporated herein by reference in its entirety.

### TECHNICAL FIELD

The present disclosure relates to a light stabilizer capable of improving the light stability of a conjugated polymer and a method for preparing the same. More particularly, the present disclosure relates to a light stabilizer which prevents photooxidation of a low band gap conjugated polymer having high photon absorptivity in the presence of light/oxygen, an organic photovoltaic cell containing the same and a method for preparing the same.

### BACKGROUND

Recently, with the concerns about depletion of fossil resources as major energy sources and environmental problems such as the greenhouse effect caused by carbon dioxide emission resulting from combustion of the fossil resources, the importance of development of environment-friendly alternative energy is increasing. In an effort to overcome these problems, various energy sources including hydraulic and wind power are being studied. Also, the solar light is being studied as a new renewable energy source that can be used unlimitedly.

A photovoltaic cell using the solar light can be largely classified into a photovoltaic cell using an inorganic material such as silicon and one using an organic material. Especially, a polymer-based organic thin-film photovoltaic cell is studied a lot for many advantages over a silicon-based inorganic photovoltaic cell, including low production cost, lightweightness, production by various methods including roll-to-roll processing and inkjet printing and production of large-sized flexible devices that can be bent freely.

Typical materials used in a photoactive layer of an organic thin-film photovoltaic cell include [4,8-bis-substituted-benzo[1,2-b:4,5′]dithiophene-2,6-diyl-alt-4-substituted-thieno[3,4-b]thiophene-2,6-diyl] (PBDTTT)-derived polymers (PTB7) (Y. Liang, Z. Xu, J. Xia, S.-T. Tsai, Y. Wu, G. Li, C. Ray, L. Yu, Adv. Energy Mater., 2010, 22, E135-E138), PBDTTT-C (H.-Y. Chen, J. Hou, S. Zhang, Y. Liang, G. Yang, Y. Yang, L. Yu, Y. Wu, G. Li, Nat. Photon. 2009, 3, 649-653), etc. It is reported that these conjugated polymers exhibit high photoconversion efficiency of 7% or greater.

However, because these conjugated polymers have very poor light stability, they result in very poor light stability of the optoelectronic devices containing them. In order to overcome these problems and to ensure light stability of high-efficiency organic photovoltaic cell devices, development of a light stabilizing additive that can be contained in a photoactive layer is necessary.

### REFERENCES OF THE RELATED ART

### Non-Patent Documents

Adv. Energy Mater., 2010, 22, E135-E138.

Nat. Photon. 2009, 3, 649-653.

### SUMMARY

The present disclosure is directed to providing an additive which prevents photooxidation of a low band gap conjugated polymer having high photon absorptivity in the presence of light/oxygen and a method for preparing the same.

The present disclosure is also directed to providing a highly stable high-efficiency organic photovoltaic cell containing the light stabilizing additive.

In an aspect, the present disclosure provides a light stabilizer having a structure of Chemical Formula 1:

R¹—Ar¹—NH—Ar²—NH—Ar³—R² Chemical Formula 1

In another aspect, the present disclosure provides a photoactive layer containing the light stabilizer according to the present disclosure.

In another aspect, the present disclosure provides an optoelectronic device containing the light stabilizer according to the present disclosure.

In another aspect, the present disclosure provides a method for preparing the light stabilizer having a structure of Chemical Formula 1.

Since the additive of the present disclosure improves light stability, it can be usefully used as a material for various organic optoelectronic devices such as an organic photovoltaic (OPV) cell, an organic photodiode (OPD), an organic thin-film transistor (OTFT), an organic light-emitting diode (OLED), etc.

### BRIEF DESCRIPTION OF DRAWINGS

FIG. 1A shows the chemical structure of the PTB7 polymer.

FIG. 1B shows the absorbance of a PTB7 polymer film and the change in absorbance with time when solar light of 100 mW/cm² intensity was radiated.

FIG. 2 shows the absorbance of a PTB7 polymer film and the change in absorbance with time when solar light of 100 mW/cm² intensity was radiated. When forming the PTB7 polymer film, 3 vol % of 1,8-diiodooctane (DIO) was added to a polymer solution.

FIG. 3 shows the absorbance of a PTB7 polymer film according to an exemplary embodiment of the present disclosure and the change in absorbance with time when solar light of 100 mW/cm² intensity was radiated. When forming the PTB7 polymer film, 3 vol % of 1,8-diiodooctane (DIO) and 1 wt % of a light stabilizer 1a based on the polymer were added to a polymer solution.

FIG. 4 shows the absorbance of a PTB7 polymer film according to an exemplary embodiment of the present disclosure and the change in absorbance with time when solar light of 100 mW/cm² intensity was radiated. When forming the PTB7 polymer film, 3 vol % of 1,8-diiodooctane (DIO) and 1 wt % of a light stabilizer 1c based on the polymer were added to a polymer solution.

FIG. 5 shows the relative light stability of a PTB7 film containing 1,8-diiodooctane (DIO), a light stabilizer 1a or a light stabilizer 1c with time. The relative light stability is determined by dividing the absorbance of the additive-containing PTB7 film by the absorbance of an additive-free PTB7 film.

FIG. 6 shows the change in photoconversion efficiency of (i) an antireflective layer/ITO glass/TiO₂/PTB7:PC₇₁BM/MoO₃/Ag device and (ii) an antireflective layer/ITO glass/TiO₂/PTB7:PC₇₁BM:1c (1 wt %)/MoO₃/Ag device under solar light of 100 mW/cm² intensity with time. The initial photoconversion efficiency of the device without an additive 1c and containing the additive was 6.52% and 6.18%, respectively.

### DETAILED DESCRIPTION OF EMBODIMENTS

Hereinafter, various aspects and exemplary embodiments of the present disclosure will be described in more detail.

In an aspect, the present disclosure provides a light stabilizer having a structure of Chemical Formula 1:

R¹—Ar¹—NH—Ar²—NH—Ar³—R² Chemical Formula 1

wherein

each of Ar¹, Ar² and Ar³, which are identical or different, is independently selected from the following structures; and

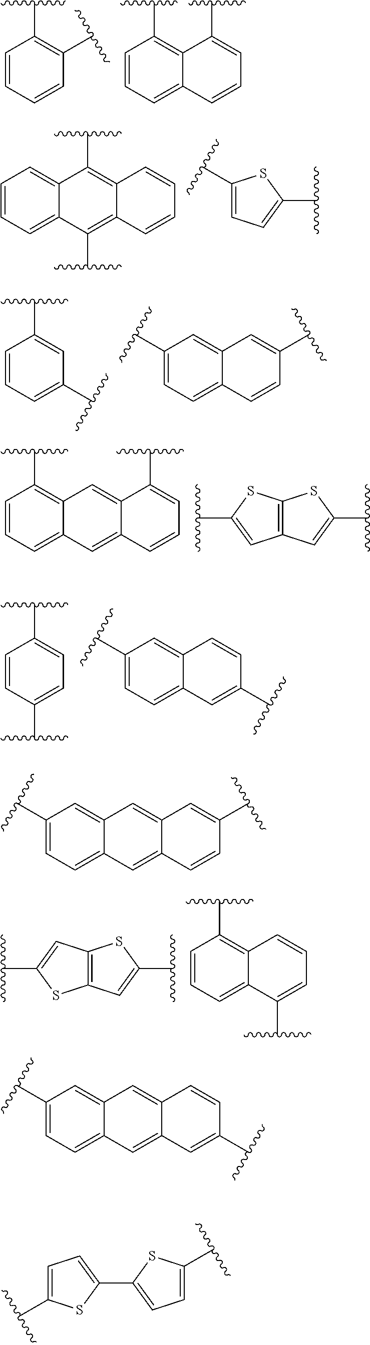

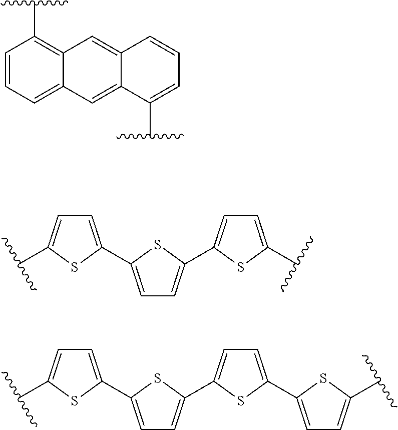 [SMILES: *c1cccc2cc3c(*)cccc3cc12 ; *c1ccc(-c2ccc(-c3ccc(*)s3)s2)s1 ; *c1ccc(-c2ccc(-c3ccc(-c4ccc(*)s4)s3)s2)s1]

each of R¹ and R², which are identical or different, is independently selected from a linear or branched C₁-C₇ alkyl group, a linear or branched C₈-C₂₀ alkyl group, a linear or branched C₁-C₇ alkoxy group and a linear or branched C₈-C₂₀ alkoxy group.

In an exemplary embodiment, the Ar¹ and the Ar³ are identical to each other.

In this case, synthesis of the material is easier and the decrease of photoconversion efficiency with time can be improved as compared to when the Ar¹ and the Ar³ are different from each other.

In another exemplary embodiment, each of the Ar¹, the Ar² and the Ar³, which are identical or different, is independently selected from

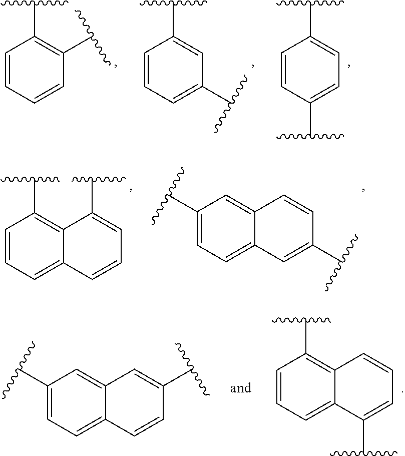

In this case, the compound itself becomes stable in the presence of light/oxygen/moisture as compared to when the Ar¹, the Ar² and the Ar³ are anthracene- thiophene- or thienothiophene-based because the HOMO level (highest occupied molecular orbital energy level) is lower.

In another exemplary embodiment, each of the Ar¹ and the Ar³, which are identical or different, is independently selected from

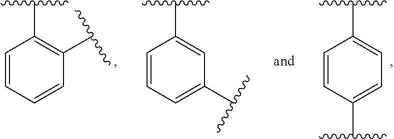 [SMILES: *c1ccccc1* ; *c1cccc(*)c1 ; *c1ccc(*)cc1]

and the Ar² is selected from

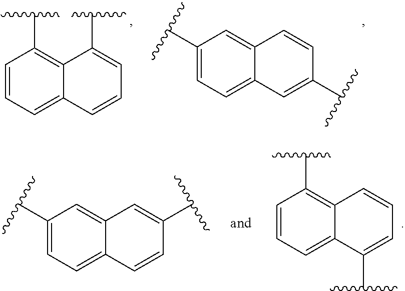 [SMILES: *c1cccc2cccc(*)c12 ; *c1ccc2cc(*)ccc2c1 ; *c1ccc2ccc(*)cc2c1 ; *c1cccc2c(*)cccc12]

In another exemplary embodiment, the Ar¹ and the Ar³ are

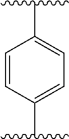 [SMILES: *c1ccc(*)cc1]

### and the Ar² is

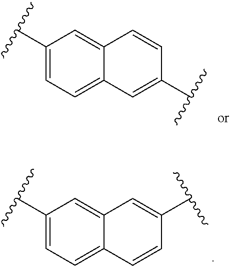 [SMILES: *c1ccc2cc(*)ccc2c1 ; *c1ccc2ccc(*)cc2c1]

The two structures are advantageous over the other structures of Chemical Formula 1 in that the adequate amount of electrons of the naphthalene structure provides high light stability.

In another exemplary embodiment, the R¹ and the R² are identical to each other.

In this case, synthesis of the compound is easier and the decrease of photoconversion efficiency with time can be improved as compared to when the R¹ and the R² are different from each other.

In another exemplary embodiment, each of the R¹ and the R², which are identical or different, is independently a linear or branched C₁-C₇ alkyl group.

When the R¹ and the R² are independently a linear or branched C₁-C₇ alkyl group, light stability can be improved as compared to when they are absent or other substituents, because of uniform mixing with a conductive material (particularly, a polymer) during film formation due to high solubility.

In another exemplary embodiment, the R¹ and the R² are the same linear or branched C₁-C₇ alkyl group.

In another exemplary embodiment, the R¹ and the R² are hexyl.

In another exemplary embodiment, the light stabilizer has one of the following structures:

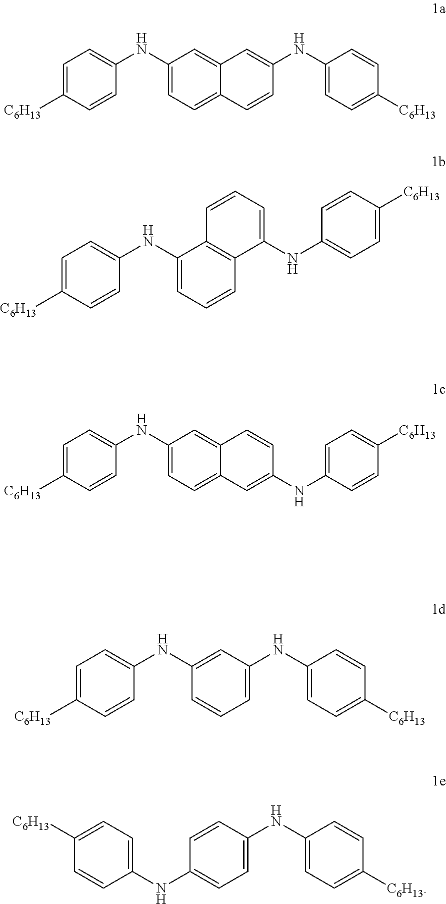

In another aspect, the present disclosure provides a photoactive layer containing a conjugated polymer and a light stabilizer according to the present disclosure.

In an exemplary embodiment, the light stabilizer may be contained in an amount of 0.1-5 wt %, specifically 0.5-3 wt %, based on the weight of the conjugated polymer.

If the light stabilizer is contained in an amount less than the lowest limit, the light stabilizing effect may be insignificant. And, if the content exceeds the highest limit, the initial characteristics of the optoelectronic device may be negatively affected (for example, the efficiency of the organic photovoltaic cell may decrease).

In another exemplary embodiment, the photoactive layer may further contain a photodissociation inhibitor.

Under some environments, the light stabilizer according to the present disclosure may exhibit light stabilizing effect only when a photodissociation inhibitor is further contained. Also, since the addition of a photodissociation inhibitor improves the light stabilizing effect under other environments, it is preferred that a photodissociation inhibitor is further contained.

However, the photodissociation inhibitor is not an essential component since the light stabilizer may exhibit light stabilizing effect under other environments even when the photodissociation inhibitor is not further contained.

For example, the photodissociation inhibitor may be 1,8-diiodooctane, 1,6-diiodohexane, 1-chloronaphthalene, 1,8-ocatnedithiol or a mixture thereof, although not being limited thereto.

In another exemplary embodiment, the photodissociation inhibitor may be contained in an amount of 1-10 vol % based on a solvent (e.g., chlorobenzene) for forming the conjugated polymer into a film.

The photodissociation inhibitor may be contained in an amount of 0.1-0.3 wt % based on 100 wt % of the conjugated polymer.

If the amount of the photodissociation inhibitor is smaller than the lowest limit, the light stabilizing effect may be insignificant. And, if it exceeds the highest limit, the uniformity and surface roughness of the formed polymer film may be unsatisfactory.

In another aspect, the present disclosure provides an optoelectronic device containing the light stabilizer according to the present disclosure. Examples of the optoelectronic device may include an organic photovoltaic cell, an organic photodiode, an organic light-emitting diode, an organic thin-film transistor, etc., although not being limited thereto.

In another aspect, the present disclosure provides a method for preparing a compound of Chemical Formula 1, including a step (A) of reacting a compound of Chemical Formula 2 with a compound of Chemical Formula 3 (see Scheme 1).

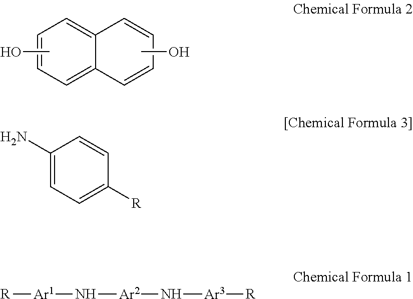

In the above chemical formulas,

Ar¹ and Ar³ are

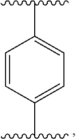 [SMILES: *c1ccc(*)cc1]

Ar² is selected from

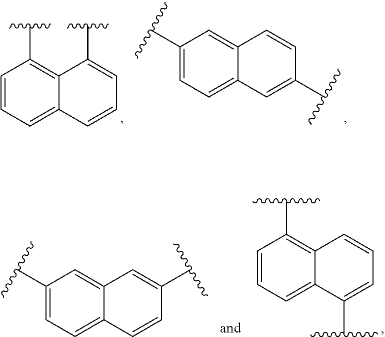 [SMILES: *c1cccc2cccc(*)c12 ; *c1ccc2cc(*)ccc2c1 ; *c1ccc2ccc(*)cc2c1 ; *c1cccc2c(*)cccc12]

and

R is selected from a linear or branched C₁-C₇ alkyl group, a linear or branched C₈-C₂₀ alkyl group, a linear or branched C₁-C₇ alkoxy group and a linear or branched C₈-C₂₀ alkoxy group.

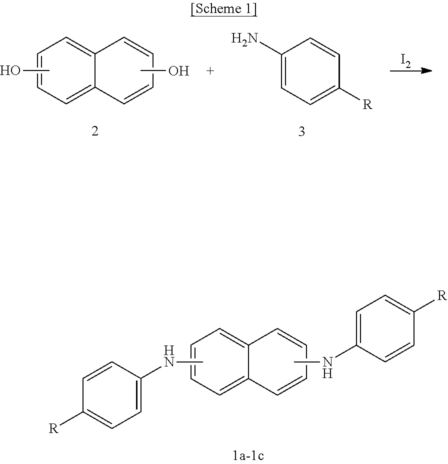

In another exemplary embodiment, the step (A) is conducted in the presence of iodine (I₁₂).

If the step (A) is conducted in the presence of iodine, it is advantageous in that the reaction is simple and purification is easy.

The reaction may be conducted by mixing the compound of Chemical Formula 2 and the compound of Chemical Formula 3 with iodine and then heating. Specifically, the reaction may be conducted by heating at 190-200° C. for 8-9 hours.

In another aspect, the present disclosure provides a method for preparing a compound of Chemical Formula 1, including a step (A′) of reacting a compound of Chemical Formula 4 with a compound of Chemical Formula 3 (see Scheme 2).

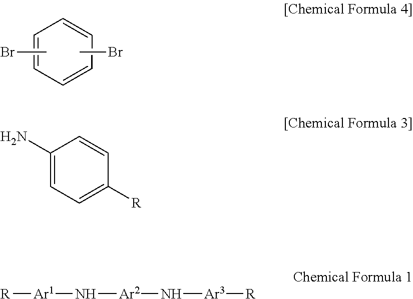

In the above chemical formulas,

Ar¹ and Ar³ are

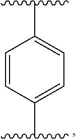 [SMILES: *c1ccc(*)cc1]

Ar² is selected from

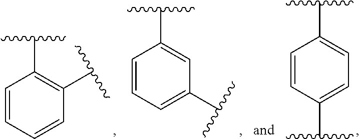

and

R is selected from a linear or branched C₁-C₇ alkyl group, a linear or branched C₈-C₂₀) alkyl group, a linear or branched C₁-C₇ alkoxy group and a linear or branched C₈-C₂₀ alkoxy group.

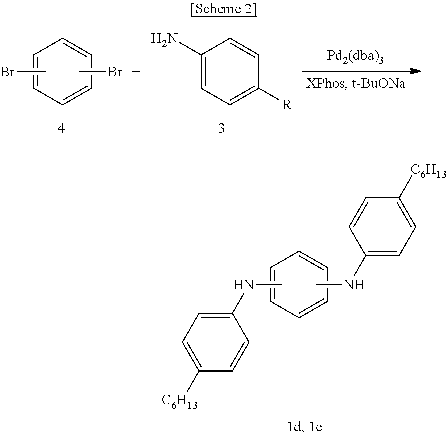

The reaction may be conducted in water, toluene, acetone, methanol, ethanol, tetrahydrofuran (THF), chlorobenzene, dimethylformamide (DMF) or a mixture solvent thereof.

In another exemplary embodiment, the step (A′) may be conducted in the presence of a palladium catalyst.

If the step (A′) is conducted in the presence of a palladium catalyst, it is advantageous in that the yield of chemical reaction is high.

In another exemplary embodiment, the palladium catalyst is selected from PdCl₂, Pd(OAc)₂, Pd(CH₃CN)₂Cl₂, Pd(PhCN)₂Cl₂, Pd₂dba₃, Pd(PPh₃)₄ and a mixture thereof.

The reaction may be conducted by dissolving the compound of Chemical Formula 3 and the compound of Chemical Formula 4 in a solvent and then adding the palladium.

Hereinafter, the present disclosure will be described in more detail through examples. However, the following examples are for illustrative purposes only and not intended to limit the scope of this disclosure.

### EXAMPLES

### Example 1-1

### Preparation of N²,N⁷-bis(4-hexylphenyl)naphthalene-2,7-diamine

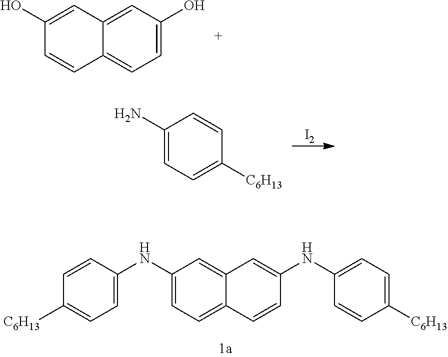

2,7-Dihydroxynaphthalene (354 mg, 2.21 mmol), I₂ (24 mg, 0.09 mmol) and 4-n-hexylaniline (1 g, 5.64 mmol) were mixed and heated at 190° C. for 8 hours. After the reaction was completed, the reaction mixture was cooled to room temperature and purified using a silica gel column (eluent: EtOAc/n-Hex=1/10) to obtain the target compound (430 mg, yield: 40.6%) (compound 1a).

¹H NMR (400 MHz, CDCl₃): δ 7.602-7.580 (d, 2H), 7.167-7.162 (d, 2H), 7.128-7.072 (m, 8H), 6.980-6.953 (dd, 2H), 5.731 (s, 2H), 2.586-2.547 (t, 4H), 1.623-1.586 (m, 4H), 1.319 (m, 12H), 0.907-0.873 (t, 6H).

### Example 1-2

### Preparation of N¹,N⁵-bis(4-hexylphenyl)naphthalene-1,5-diamine

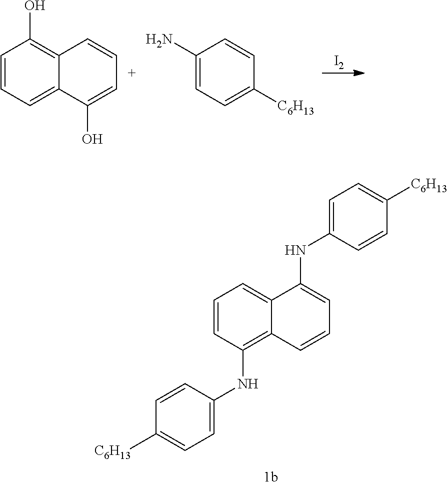

1,5-Dihydroxynaphthalene (354 mg, 2.21 mmol), I₂ (24 mg, 0.09 mmol) and 4-n-hexylaniline (1 g, 5.64 mmol) were mixed and heated at 200° C. for 8 hours. After the reaction was completed, the reaction mixture was cooled to room temperature and purified using a silica gel column (eluent: EtOAc/n-Hex=1/10) to obtain the target compound (63 mg, yield: 6.0%) (compound 1b).

¹H NMR (400 MHz, CDCl₃): δ 7.668-7.646 (dd, 2H), 7.370-7.255 (m, 4H), 7.115-7.093 (d, 4H), 7.008-6.987 (dt, 4H), 5.933 (s, 2H), 2.585-2.546 (t, 4H), 1.642-1.567 (m, 4H), 1.321 (m, 12H), 0.908-0.874 (t, 6H).

### Example 1-3

### Preparation of N²,N⁶-bis(4-hexylphenyl)naphthalene-2,6-diamine

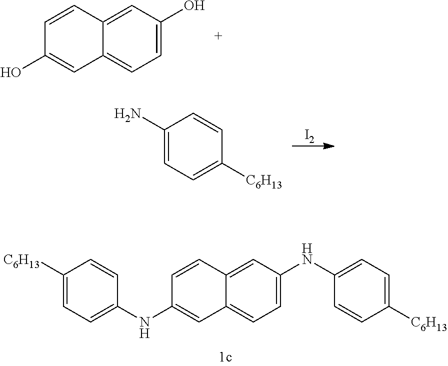

2,6-Dihydroxynaphthalene (500 mg, 3.21 mmol), I₂ (31 mg, 0.12 mmol) and 4-n-hexylaniline (1.37 g, 7.73 mmol) were mixed and heated at 190° C. for 8 hours. After the reaction was completed, the reaction mixture was cooled to room temperature and purified using a silica gel column (eluent: EtOAc/n-Hex=1/5) to obtain the target compound (86 mg, yield: 5.6%) (compound 1c).

¹H NMR (400 MHz, DMSO): δ 8.054 (s, 2H), 7.564-7.542 (d, 2H), 7.323-7.317 (d, 2H), 7.162-7.135 (dd, 2H), 7.079-7.027 (m, 8H), 2.509-2.473 (m, 4H), 1.556-1.520 (m, 4H), 1.286-1.276 (m, 12H), 0.879-0.845 (t, 6H).

### Example 1-4

### Preparation of N¹,N³-bis(4-hexylphenyl)benzene-1,3-diamine

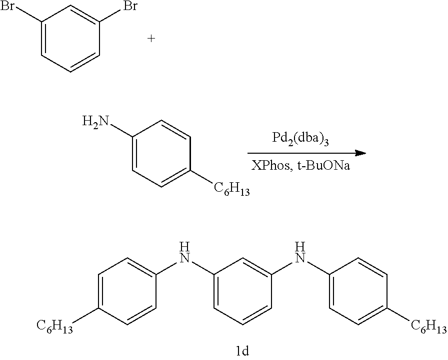

1,3-Dibromobenzene (0.5 g, 2.1 mmol) was dissolved in 1,4-dioxane, mixed with 4-hexylaniline (0.88 mL, 4.5 mmol), Pd₂(dba)₃ (0.061 mg, 0.1 mmol), XPhos (0.1 g, 0.21 mmol) and t-BuONa (0.6 g, 6.4 mmol) and then heated at 100° C. for 18 hours. After the reaction was completed, the reaction mixture was cooled to room temperature and water was added. The mixture was extracted with MC, dried with Na₂SO₄, concentrated and suspended in n-hexane to obtain the target compound (0.42 g, yield: 47%) (compound 1d).

¹H NMR (400 MHz, CDCl₃): δ 7.100-7.073 (m, 5H), 7.028-7.007 (m, 4H), 6.682-6.672 (t, 1H), 6.560-6.534 (m, 2H), 5.568 (s, 2H), 2.572-2.533 (t, 4H), 1.612-1.576 (m, 4H), 1.322-1.318 (m, 12H), 0.911-0.877 (t, 6H).

### Example 1-5

### Preparation of N¹,N⁴-bis(4-hexylphenyl)benzene-1,4-diamine

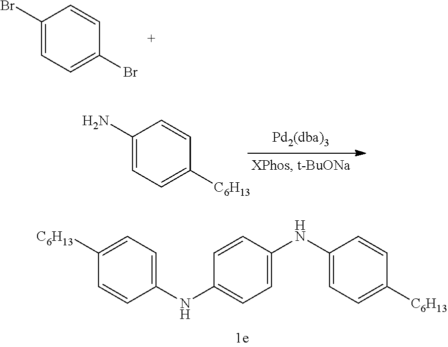

1,4-Dibromobenzene (0.5 g, 2.1 mmol) was dissolved in 1,4-dioxane, mixed with 4-hexylaniline (0.88 mL, 4.5 mmol), Pd₂(dba)₃ (0.061 mg, 0.1 mmol), XPhos (0.1 g, 0.21 mmol) and t-BuONa (0.6 g, 6.4 mmol) and then heated at 100° C. for 18 hours. After the reaction was completed, the reaction mixture was cooled to room temperature and water was added. The mixture was extracted with MC, dried with Na₂SO_{4,} concentrated and suspended in MeOH to obtain the target compound (0.68 g, yield: 75%) (compound 1e).

¹H NMR (400 MHz, CDCl₃): δ 7.065-7.044 (d, 4H), 7.006 (s, 4H), 6.925-6.905 (d, 2H), 5.464 (s, 2H), 2.555-2.516 (t, 4H), 1.600-1.547 (m, 4H), 1.312-1.308 (m, 12H), 0.905-0.872 (t, 6H).

### Comparative Example 2-1

### Preparation of Photoactive Layer (Photoconversion Layer)

An ITO substrate was washed with isopropyl alcohol for 10 minutes, with acetone for 10 minutes and then with isopropyl alcohol for 10 minutes and then dried before use. A solution of PTB7 (10 mg) and a light stabilizer (1 mg) dissolved in a chlorobenzene solvent (1 mL) was spin coated on the dried ITO substrate at a rate of 1,500 rpm.

### Comparative Example 2-2

### Preparation of Photoactive Layer

An ITO substrate was washed with isopropyl alcohol for 10 minutes, with acetone for 10 minutes and then with isopropyl alcohol for 10 minutes and then dried before use. A solution of PTB7 (10 mg) and a light stabilizer (1 mg) dissolved in a chlorobenzene solvent (1 mL) was spin coated on the dried ITO substrate at a rate of 1,500 rpm.

### Comparative Example 2-2

### Preparation of Photoactive Layer

An ITO substrate was washed with isopropyl alcohol for 10 minutes, with acetone for 10 minutes and then with isoproply alcohol for 10 minutes and then dried before use. A solution of PTB7 (10 mg) dissolved in a 97:3 (v/v) mixture solvent (1 mL) of chlorobenzene and 1,8-diiodooctane (DIO) was spin coated on the dried ITO substrate at a rate of 1,500 rpm.

### Examples 2-1 and 2-2

### Preparation of Photoactive Layer

An ITO substrate was washed with isopropyl alcohol for 10 minutes, with acetone for 10 minutes and then with isopropyl alcohol for 10 minutes and then dried before use. A solution of PTB7 (10 mg) and the compound 1a (1 mg) prepared in Example 1-1 or and the compound 1c (1 mg) prepared in Example 1-3 dissolved in a 97:3 (v/v) mixture solvent (1 mL) of chlorobenzene and 1,8-diiodooctane (DIO) was spin coated on the dried ITO substrate at a rate of 1,500 rpm. The finally formed photoactive layer was found to contain the DIO in an amount of about 0.2 wt % (Example 2-1 and Example 2-2) based on 100 wt % of the conjugated polymer.

### Comparative Example 3-1

### Preparation of ITO/TiO₂/PTB7:PC₇₁BM (1:1.5)/MoO₃/Ag Photovoltaic Cell

An ITO substrate was washed with isopropyl alcohol for 10 minutes, with acetone for 10 minutes and then with isopropyl alcohol for 10 minutes and then dried before use. A solution of TiO₂ nanoparticles in ethanol was spin coated on the dried ITO substrate, which was then dried at 60° C. for 10 minutes. A solution of 1:1.5 (w/w) of PTB7 (10 mg) and PC₇₁BM (15 mg) dissolved in a 97:3 (v/v) mixture solvent (1 mL) of chlorobenzene and 1,8-diiodooctane (DIO) was spin coated on the dried substrate at a rate of 1,500 rpm. Then, a photovoltaic cell device was completed by depositing a 4-nm thick MoO₃ layer and a 100-nm thick Ag electrode. Finally, an antireflective film was adhered on the outside of the transparent electrode of the device.

### Example 3-1

### Preparation of ITO/TiO₂/PTB7:PC₇₁BM (1:1.5)+1c (0.1)/MoO₃/Ag Photovoltaic Cell

A photovoltaic cell was prepared in the same manner as in Comparative Example 3-1, except that a solution of polymer PTB7 (10 mg), PC₇₁BM (15 mg) and the compound 1c (1 mg) prepared in Example 1-3 dissolved in a 97:3 (v/v) mixture solvent (1 mL) of chlorobenzene and 1,8-diiodooctane (DIO) was used instead of the solution of 1:1.5 (w/w) of PTB7 (10 mg) and PC₇₁BM (15 mg) dissolved in a 97:3 (v/v) mixture solvent (1 mL) of chlorobenzene and 1,8-diiodooctane (DIO).

### Comparative Test Examples 1-1 and 1-2 and Test Examples 1-1 and 1-2

### Measurement of Optical Band Gap

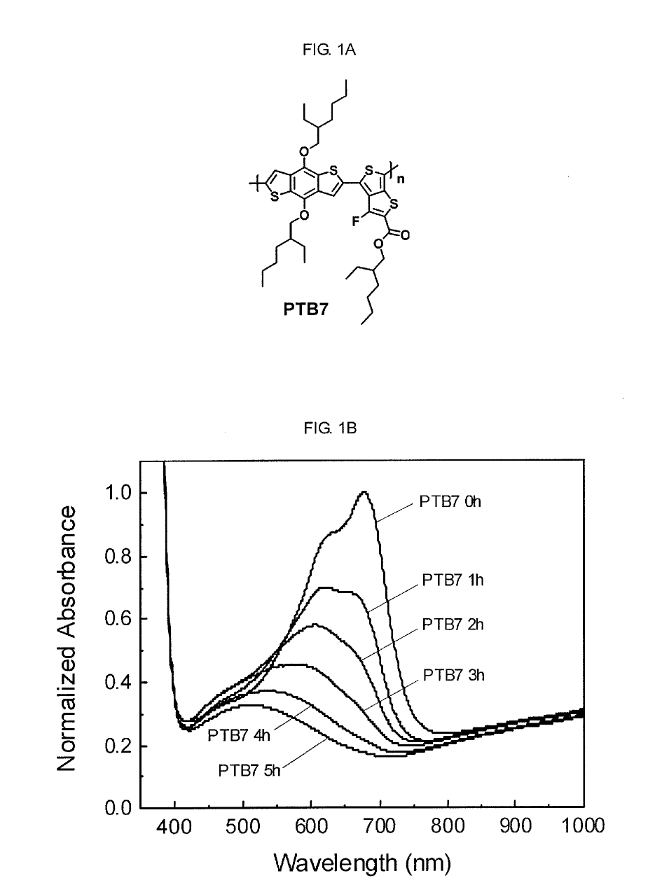

Measurement was made for the photoactive films prepared in Comparative Examples 2-1 and 2-2 and Examples 2-1 and 2-2 using a UV-Vis spectrometer. (i) Absorbance and (ii) progress of photodissociation of the PTB7 polymer film (i.e., change in absorption spectrum) were measured as functions of time. The result is shown in FIGS. 1-4.

As seen from FIG. 1, the conductive polymer PTB7 showed maximum absorption around 700 nm and was found to absorb light up to 750 nm. When solar light of 100 mW/cm² intensity was radiated, the absorbance of the PTB7 film decreased rapidly with time in the longer wavelength region (600-750 nm).

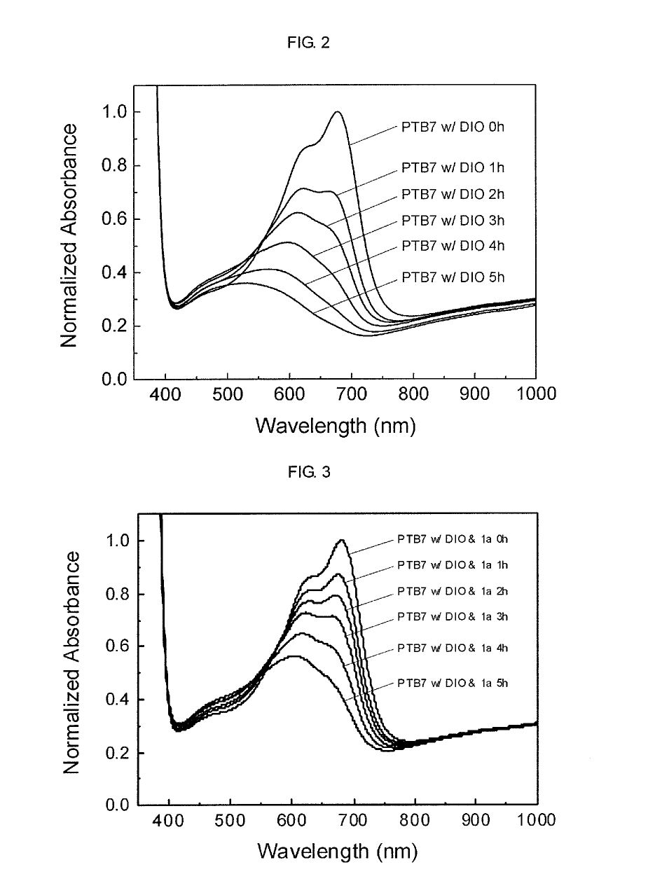

And, as seen from FIG. 2, when only the photodissociation inhibitor DIO was added, the improvement in the light stability of the PTB7 film was hardly observed.

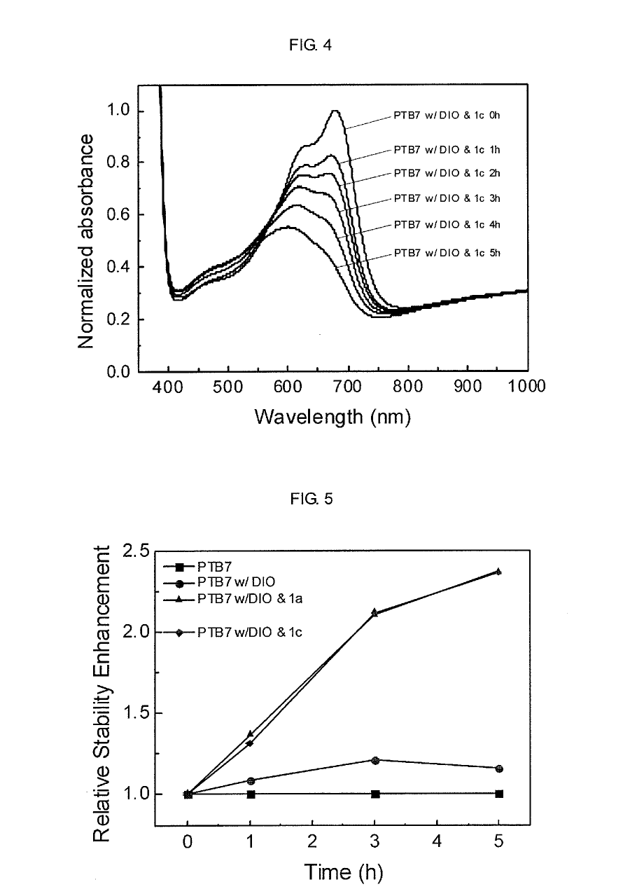

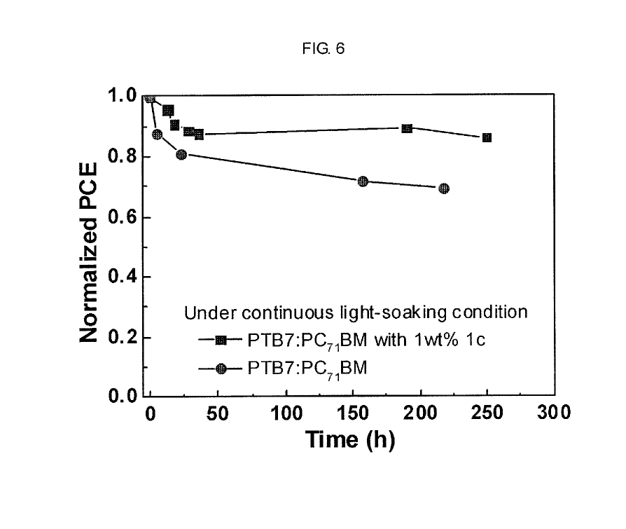

In contrast, as seen from FIG. 3 and FIG. 4, when the light stabilizer prepared in Example 1-1 or 1-3 was added in a small amount (1 wt %) (Example 2-1 and Example 2-2, respectively), the decrease in absorbance with time was greatly reduced.

Although the data were not presented in the present disclosure, the compounds la and 1c provided superior light stabilizing effect as compared to the compounds 1b, 1d and 1e.

Accordingly, the additive which enhances the light stability of the conductive polymer can be usefully used to improve the reliability of an organic photovoltaic cell and can also be usefully used as a material for an organic optoelectronic device selected from an organic photodiode (OPD), an organic light-emitting diode (OLED) and an organic thin-film transistor (OTFT).

### Comparative Test Example 2-1 and Test Example 2-1

### Evaluation of Performance of Organic Photovoltaic Cell

The performance of organic photovoltaic cell devices prepared in Comparative Example 3-1 and Example 3-1 was evaluated under solar light of 100 mW/cm² intensity. Fill factor and energy conversion efficiency were calculated according to Equation 1 and Equation 2. The change in energy conversion (photoconversion) efficiency is shown in FIG. 6.

As seen from FIG. 6, the photovoltaic cell devices prepared in Comparative Example 3-1 and Example 3-1 exhibit similar initial photoconversion efficiency as 6.52% and 6.18%, respectively. However, it can be seen that the photovoltaic cell device prepared in Example 3-1 shows significantly improved ability of maintaining the initial efficiency as compared to that prepared in Comparative Example 3-1.

Fill factor=(Vₘₚ×Iₘₚ)/(V_{oc}×I_{sc}) [Equation 1]

where Vₘₚ is the voltage at the maximum power point, Iₘₚ is the current at the maximum power point, V_{oc} is the open circuit voltage and I_{sc} is the short circuit current.

Energy conversion efficiency (%)=Fill factor×(J_{sc}×V_{oc})/100 Equation 2

where J_{sc} is the short circuit current density and V_{oc} is the open circuit voltage.

This result confirms that the light stability improving additive of the present disclosure is suitable for use in an organic photovoltaic cell and improves the light stability of the organic photovoltaic cell. Accordingly, the light stabilizer according to the present disclosure can be usefully used as a stability improving additive of an organic photovoltaic cell device using a conductive polymer and can also be usefully used as a material for an organic optoelectronic device using a conjugated conductive polymer, such as an organic photodiode (OPD), an organic thin-film transistor (OTFT), an organic light-emitting diode (OLED), etc.

What is claimed is:

1. A light stabilizer having a structure of Chemical Formula 1:
R¹—Ar¹—NH—Ar²—NH—Ar³—R² Chemical Formula 1
wherein
each of Ar¹,Ar² and Ar³, which are identical or different, is independently selected from the following structures; and 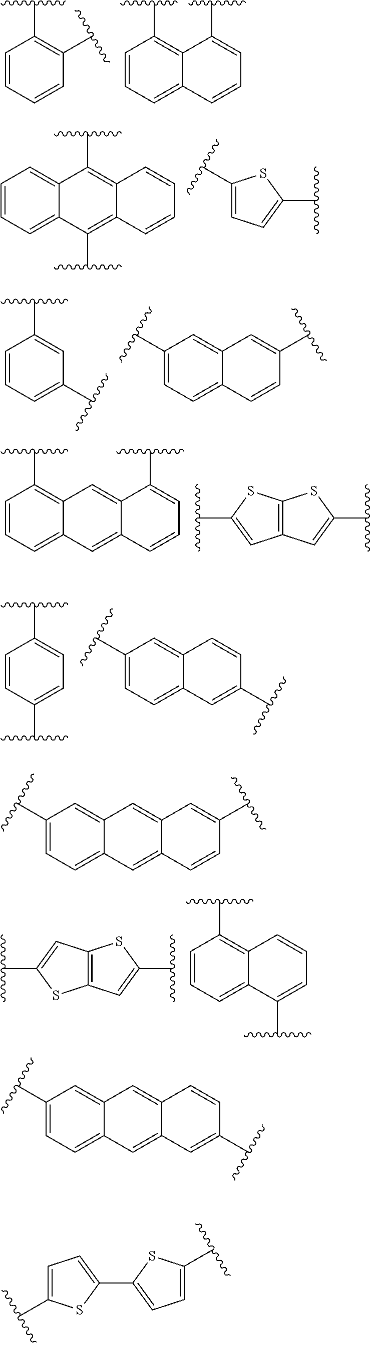 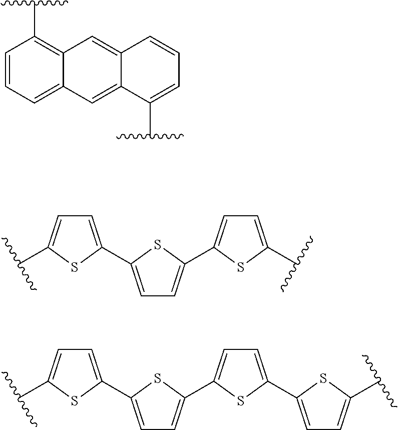 [SMILES: *c1cccc2cc3c(*)cccc3cc12 ; *c1ccc(-c2ccc(-c3ccc(*)s3)s2)s1 ; *c1ccc(-c2ccc(-c3ccc(-c4ccc(*)s4)s3)s2)s1]
each of R¹ and R², which are identical or different, is independently selected from a linear or branched C₁-C₇ alkyl group, a linear or branched C₈-C₂₀ alkyl group, a linear or branched C₁-C₇ alkoxy group and a linear or branched C₈-C₂₀ alkoxy group.

2. The light stabilizer according to claim 1, wherein the Ar³ and the Ar³ are identical to each other.

3. The light stabilizer according to claim 1, wherein each of the Ar³, the Ar² and the Ar³, which are identical or different, is independently selected from

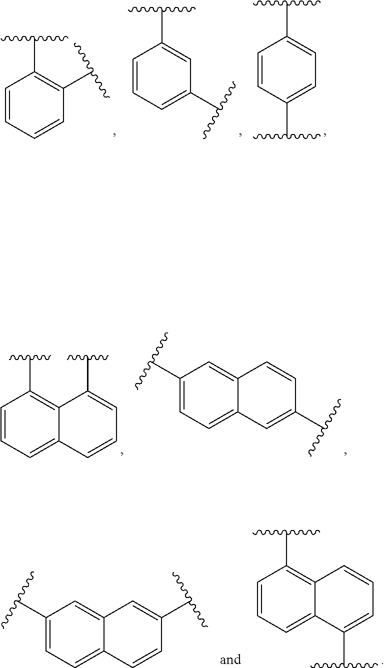

4. The light stabilizer according to claim 1, wherein each of the Ar¹ and the Ar³, which are identical or different, is independently selected from 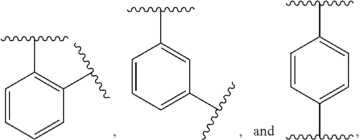
and the A² is selected from

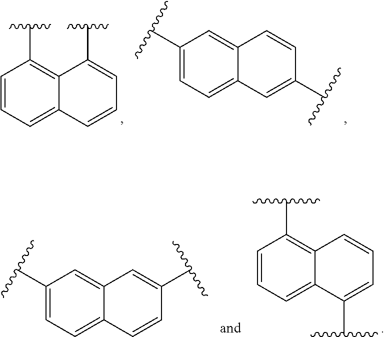 [SMILES: *c1cccc2cccc(*)c12 ; *c1ccc2cc(*)ccc2c1 ; *c1ccc2ccc(*)cc2c1 ; *c1cccc2c(*)cccc12]

5. The light stabilizer according to claim 1, wherein the Ar¹ and the Ar³ are 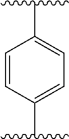 [SMILES: *c1ccc(*)cc1]
and the Ar² is

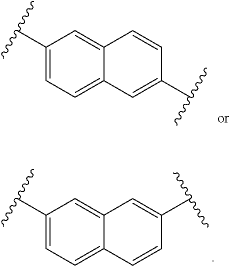 [SMILES: *c1ccc2cc(*)ccc2c1 ; *c1ccc2ccc(*)cc2c1]

6. The light stabilizer according to claim 1, wherein the R¹ and the R² are identical to each other.

7. The light stabilizer according to claim 1, wherein each of the R¹ and the R², which are identical or different, is independently a linear or branched C₁-C₇ alkyl group.

8. The light stabilizer according to claim 1, wherein the R¹ and the R² are the same linear or branched C₁-C₇ alkyl group.

9. The light stabilizer according to claim 1, wherein the R¹ and the R² are hexyl.

10. The light stabilizer according to claim 1, wherein the light stabilizer has one of the following structures:

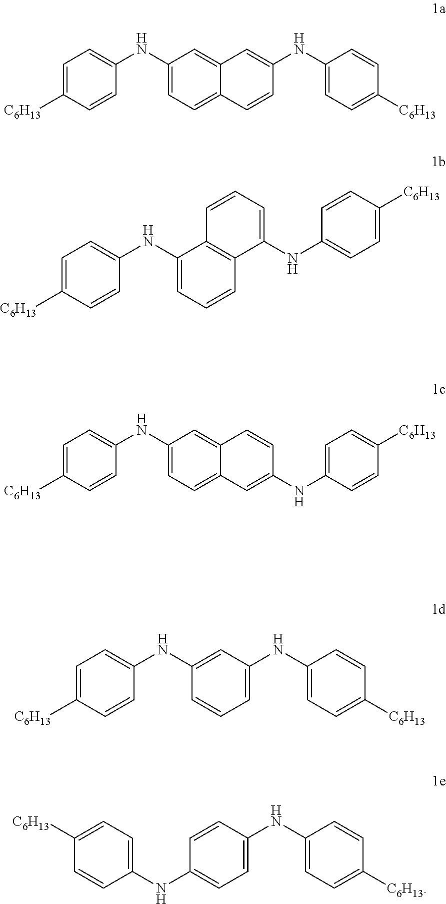

11. A photoactive layer comprising a conjugated polymer and the light stabilizer according to claim 1.

12. The photoactive layer according to claim 11, wherein the light stabilizer is comprised in an amount of 0.1-5 wt % based on the weight of the conjugated polymer.

13. The photoactive layer according to claim 11, wherein the photoactive layer further comprises a photodissociation inhibitor.

14. The photoactive layer according to claim 13, wherein the photodissociation inhibitor is selected from 1,8-diiodooctane, 1,6-diiodohexane, 1-chloronaphthalene, 1,8-ocatnedithiol and a mixture thereof.

15. The photoactive layer according to claim 13, wherein the photodissociation inhibitor is comprised in an amount of 0.1-0.3 wt % based on the weight of the conjugated polymer.

16. An optoelectronic device comprising the light stabilizer according to claim 1, wherein the optoelectronic device is selected from an organic photovoltaic cell, an organic photodiode, an organic light-emitting diode and an organic thin-film transistor.
